(19) United States
(12) Patent Application Publication
(10) Pub. No.: US 2008/0021496 A1
(43) Pub. Date: Jan. 24, 2008

(54) Embedded earlobe expander

(76) Inventor: Fernando Omar Narvaez, Atlanta, GA (US)

Correspondence Address: Fernando Omar Narvaez, 436 Lark Ave, Miami Springs, FL, 33166, US

(21) Appl. No.: 11/483,227

(22) Filed: Jul. 10, 2006

(51) Int. Cl. A61M 29/00

(52) U.S. Cl. 606/198

(57) ABSTRACT

The Embedded Earlobe Expander (EEE) differs from the solid cylindrical earlobe-stretching devices, or “plugs,” presently used in that its outer perimeter has partitioned arcs connected by moveable screws to the center of the cylinder. Like a conventional solid plug, the EEE is inserted into an existent earlobe aperture, but the EEE's adjustable design allows the wearer to adjust the size of the device to however small or large is desired according to the choice of the wearer. The wearer adjusts the screws, either before or during wear, to his or her choice of stretching pressure or wearing tension. The wearer's ability to manipulate the screws to any desired size offers several advantages over the standard practice of graduated plug sizes, such as a more gradual increase in size, a wider range of sizes, and the added ability to decrease the size for easy removal.

### BACKGROUND OF THE INVENTION

The Embedded Earlobe Expander (EEE) pertains to the field of body piercing, specifically the practice of inserting cylindrical “plugs” into an earlobe hole that is larger than the conventional ear piercing, a practice called “ear stretching.” Conventionally in ear stretching, a large hole is pierced into the wearer's ear/s, usually beginning with 14 mm, and then a cylindrical plug is inserted through the hole, thereby stretching a hole in the earlobe. When the wearer desires to stretch the earlobe hole more, the process as it is practiced now dictates that the wearer remove the initial plugs and replace them with cylindrical plugs of a larger circumference than that of the predecessor. This sudden insertion of cylinders with a circumference larger than the existent aperture stretches the earlobe hole abruptly and painfully. Through its wearer-operated stretch design, the Embedded Earlobe Expander offers a more gradual method of ear stretching that is controlled by the wearer, allowing the wearer more control and more options in the process of stretching. The inventor's search of the USPTO's databases revealed no device similar in design and purpose.

### BRIEF SUMMARY OF THE INVENTION

The Embedded Earlobe Expander (EEE) differs from the solid cylindrical earlobe-stretching plugs in that its outer perimeter has partitioned arcs connected by moveable screws to the center of the cylinder. Like a conventional solid plug, the EEE is inserted into an existent earlobe hole, but the EEE's adjustable design allows the wearer to manipulate the size of the device to however small or large is desired according to the choice of the wearer. The wearer adjusts the screws, either before or during wear, to his or her choice of stretching pressure or wearing tension. The wearer's ability to manipulate the screws to any desired size offers several advantages over the standard practice of graduated plug sizes:

1) since the Embedded Earlobe Expander adjusts to a range of sizes, the wearer only needs to purchase one pair of EEEs, as opposed to a pair of solid cylindrical plugs in each of the desired sizes;

2) because the EEE is continuously adjustable, the wearer has a larger choice of sizes than with the predetermined sizes of solid plugs;

3) since the wearer can adjust the EEE as much or as little as desired, the wearer has more control over the ear stretching process, thus controlling the amount of pain experienced by adjusting the screws;

4) because the wearer can gradually adjust the EEE, insertion of the device will be less abrupt than standard practice, as the circumference of the EEE can be adjusted to less than that of the existent earlobe hole's upon insertion;

5) since the size of the EEE is adjustable, if the wearer desires to remove the device before stretching is complete, the wearer can decrease the circumference of the device by adjusting the screws, thus providing an easier and less painful removal than a solid ear plug by decreasing the size of the EEE to a circumference smaller than that of the earlobe's existent hole.

The main objective of the invention is to provide a more varied and more controlled ear stretching experience, options that offer an alternative to the standard process of ear stretching.

### BRIEF DESCRIPTION OF SEVERAL VIEWS OF THE DRAWING

The following list briefly describes the numbered drawings attached:

FIG. 1: Embedded Earlobe Expander having a tripartite arc model variation, shown with screws in an open or expanded position;

FIG. 2: EEE having a tripartite arc model variation, shown with screws in a closed or non-expanded position;

FIG. 3: EEE having a quadripartite arc model variation, shown with screws in an open or expanded position;

FIG. 4: EEE having a quadripartite arc model variation, shown with screws in a closed or non-expanded position.

### DETAILED DESCRIPTION OF THE INVENTION

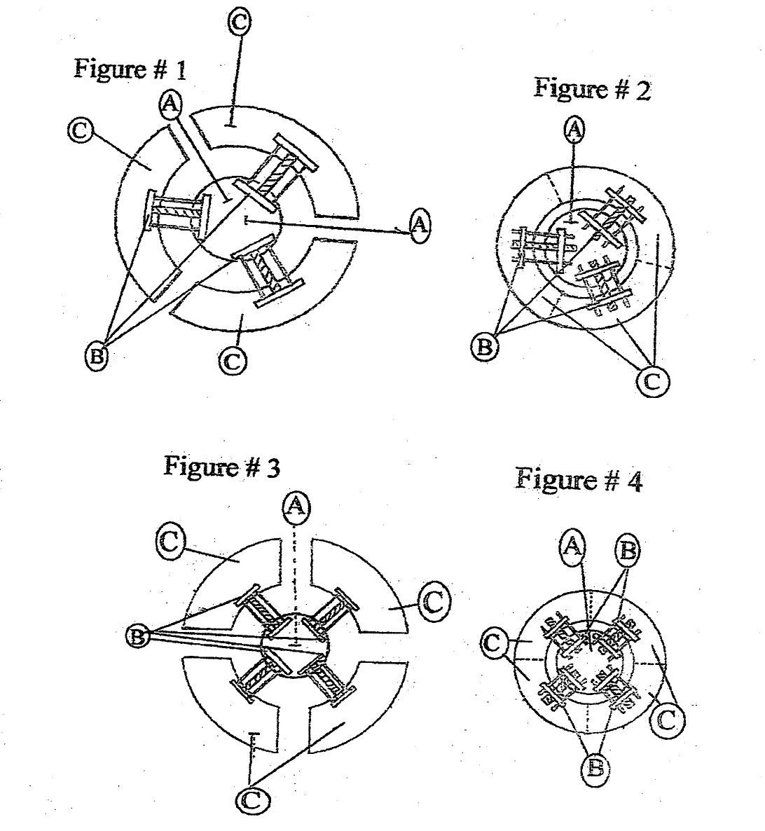

A cylinder of as much or more than earlobe width composed of materials non-reactive to skin has a center (FIG. 1A, FIG. 2A, FIG. 3A, FIG. 4A) to which are attached as many expandable screws (FIG. 1B, FIG. 2B, FIG. 3B, FIG. 4B) as necessary to control the number of partitioned concentric arcs (FIG. 1C, FIG. 2C, FIG. 3C, FIG. 4C) on the outer area of the cylinder's center. The expandable screws connect the center to the arcs, and can be expanded for the purpose of stretching or maintaining an aperture in the earlobe. This invention entitled the Embedded Earlobe Expander differs from solid, non-expandable cylindrical “plugs” used presently in the ear stretching practice in that it has the possibility to expand, with the expansion being controlled by the wearer and having the ability to increase or decrease in circumference as gradually or as quickly as the wearer desires.

The embodiments of the invention in which an exclusive property or privilege is claimed are defined as follows:

1. I claim an embedded earlobe expansion device consisting of a cylindrical form;
wherein the outer perimeter consists of partite arcs;
wherein the arcs contain expandable screws which connect the arcs concentrically to the center of the cylinder for the purpose of expanding an existent aperture within the wearer's earlobe.
